(19) United States
(12) Patent Application Publication
(10) Pub. No.: US 2016/0102282 A1
(43) Pub. Date: Apr. 14, 2016

(54) APPARATUS FOR APPLYING ELECTRIC FIELD

(71) Applicant: Samsung Electronics Co., Ltd., Suwon-si (KR)

(72) Inventors: Younsuk CHOI, Yongin-si (KR); Minseok KIM, Yongin-si (KR)

(21) Appl. No.: 14/746,799

(22) Filed: Jun. 22, 2015

(30) Foreign Application Priority Data
Oct. 10, 2014 (KR) 10-2014-0136995

(51) Int. Cl. C12M 1/42

(52) U.S. Cl. CPC C12M 35/02

(57) ABSTRACT

An apparatus for applying an electric field includes: a base, on which an internal region is defined, a plurality of electrodes disposed to face each other in parallel in the internal region of the base, and a sample plate disposed between the plurality of electrodes.

### CROSS-REFERENCE TO RELATED APPLICATION

This application claims priority to Korean Patent Application No. 10-2014-0136995 filed on Oct. 10, 2014, and all the benefits accruing therefrom under 35 U.S.C. §119, the content of which in its entirety is herein incorporated by reference.

### BACKGROUND

(a) Field

The invention relates to an apparatus for applying an electric field that applies the electric field to cells.

(b) Description of the Related Art

As an electrical stimulation apparatus for applying an electric stimulus to cells, various technologies have been developed from micro electro mechanical system (“MEMS”) devices for basic research to macro scale devices for producing cells having specific genetic traits.

An electrical stimulation apparatus for applying an electric stimulus to a multi-well array through a wire electrode may be used to simultaneously perform various electrical stimulation experiments. However, in such an apparatus, a uniform electric field may not be effectively applied to the cells, or separation between the apparatus and the cells may not be effectively performed. As a result, it may be difficult to exclude a stimulation factor in the experiment.

Such MEMS electrical stimulation apparatuses may include an apparatus which is developed to observe an effect of a direct-current (“DC”) electric field transferred through the wire on behavior of the cells in a micro channel. Further, it is known to use an electrical stimulation apparatus for applying an electric stimulus to cells in a Petri dish through the wire electrode. In addition, an electrical stimulation apparatus for applying an electric pulse to a suspended cell through an aluminum electrode has been used as an apparatus for electroporation.

### SUMMARY

In a conventional apparatus developed in order to observe an effect of a direct-current (“DC”) electric field transferred through the wire on behavior of the cells in a micro channel, it may be difficult to obtain lots of cells which are electrically stimulated once and it may not be easy to separate the apparatus from the cells. In a conventional electrical stimulation apparatus for applying an electric stimulus to cells in a Petri dish through the wire electrode, it may be difficult to uniformly apply the electric field to the cells and it may be difficult to separate the cells from the apparatus. In a conventional apparatus for electroporation, since the cells are suspended for a short time, the electric stimulus may not be applied for a long time and an operation may not be effectively performed.

Exemplary embodiments of the invention provide electrical stimulation method and apparatus which applies a uniform electric stimulus to cells, allows cells, to which an electric stimulus is applied, to be easily and selectively separated from the apparatus, excludes another chemical or physical stimulation factor other than the electric stimulus. In such embodiment, an operation, extending the apparatus, and freely setting a time for applying the electric stimulus may be effectively and efficiently set.

An exemplary embodiment of the invention provides an apparatus for applying an electric field including: a base on which an internal region is defined; a plurality of electrodes disposed to face each other in parallel in the internal region of the base; and a sample plate disposed between the plurality of electrodes.

In an exemplary embodiment, uniformity of the electric field formed between the plurality of electrodes may be about 90% or more.

In an exemplary embodiment, the plurality of electrodes may have a cuboid shape.

In an exemplary embodiment, the plurality of electrodes may include three or more electrodes, and the sample plate may include two or more sample plates.

In an exemplary embodiment, a relationship between a distance between the plurality of electrodes and a width of the sample plate may satisfy the following inequality: 1≦T/W≦2, where T denotes a distance between two adjacent electrodes of the plurality of electrodes, and W denotes the width of the sample plate disposed between the two adjacent electrodes.

In an exemplary embodiment, a relationship between a length of each of the plurality of electrodes and a length of the sample plate may satisfy the following inequality: 1≦L/I≦2, where L denotes the length of each of the plurality of electrodes, and I denotes the length of the sample plate.

In an exemplary embodiment, a groove may be defined on the base, and the sample plate may be disposed in the groove.

In an exemplary embodiment, a plurality of grooves may be defined on the base, and the plurality of electrodes may be disposed in the plurality of grooves, respectively.

In an exemplary embodiment, the base may include polystyrene, polycarbonate, glass, or crystal with ensured bio-compatibility.

In an exemplary embodiment, the plurality of electrodes may be plated with gold or platinum.

In an exemplary embodiment, the plurality of electrodes may include at least one of gold, platinum, and stainless steel.

In an exemplary embodiment, the apparatus may further include a cover unit which fixes the plurality of electrodes thereto and is coupled to the base such that the plurality of electrodes is maintained at a predetermined height with respect to the base, in which the height of the plurality of electrodes may be greater than or equal to the height of the internal region of the base.

In an exemplary embodiment, the apparatus may further include a cooling device which discharges heat in the internal region of the base to outside.

In an exemplary embodiment, the cooling device may include a heat sink disposed below the base.

According to exemplary embodiments of the invention, applying a uniform electric field to the cells, stably separating the cells from the base after the electric field is applied, and applying the uniform electric field for a desired time without a time limit may be effectively preformed. In such embodiments, simultaneously applying the electric field to the plurality of cell regions may be effectively performed because an operation with the apparatus may be effectively expanded and the temperature rise of the culture solution according to joule heating may be effectively suppressed.

### BRIEF DESCRIPTION OF THE DRAWINGS

These and/or other features of the invention will become apparent and more readily appreciated from the following detailed description of embodiments thereof, taken in conjunction with the accompanying drawings, in which:

FIGS. 1A to 1C are plan and cross-sectional views of an exemplary embodiment of an apparatus for applying an electric field according to the invention;

FIGS. 2A to 2C are perspective and cross-sectional views of exemplary embodiments of the apparatus for applying an electric field according to the invention;

FIGS. 3A and 3B are cross-sectional views illustrating an alternative exemplary embodiment of the apparatus for applying an electric field according to the invention;

FIG. 4 is a diagram illustrating another alternative exemplary embodiment of the apparatus for applying an electric field according to the invention;

FIGS. 5A and 5B are plan and front views of an exemplary embodiment of the apparatus for applying an electric field according to the invention;

FIG. 6A is a diagram illustrating distribution of the electric field which is applied by an exemplary embodiment of the apparatus for applying an electric field illustrated in FIGS. 5A and 5B;

FIG. 6B is an enlarged view of the portion A in FIG. 6A;

FIGS. 7A and 7B are plan and front views of another exemplary embodiment of the apparatus for applying an electric field according to the invention;

FIG. 8A is a diagram illustrating distribution of the electric field which is applied by an exemplary embodiment of the apparatus for applying an electric field illustrated in FIGS. 7A and 7B; and

FIG. 8B is an enlarged view of the portion B in FIG. 8A.

### DETAILED DESCRIPTION

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Hereinafter, exemplary embodiments of the invention will be described in further detail with reference to the accompanying drawings.

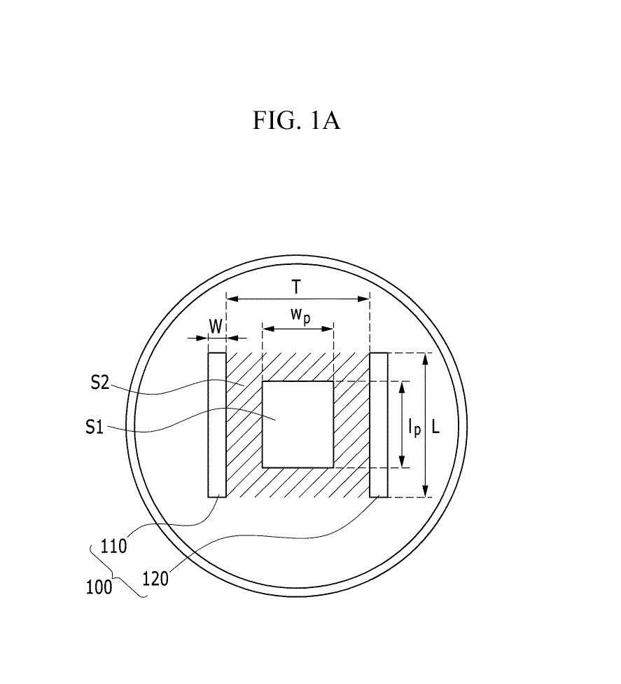

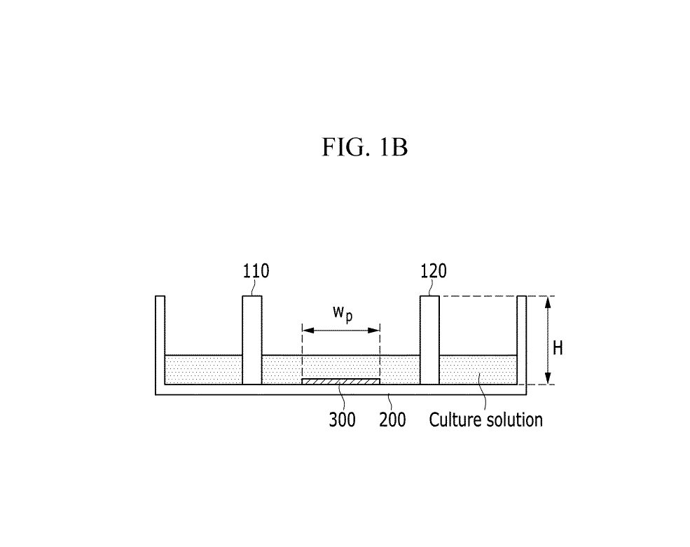

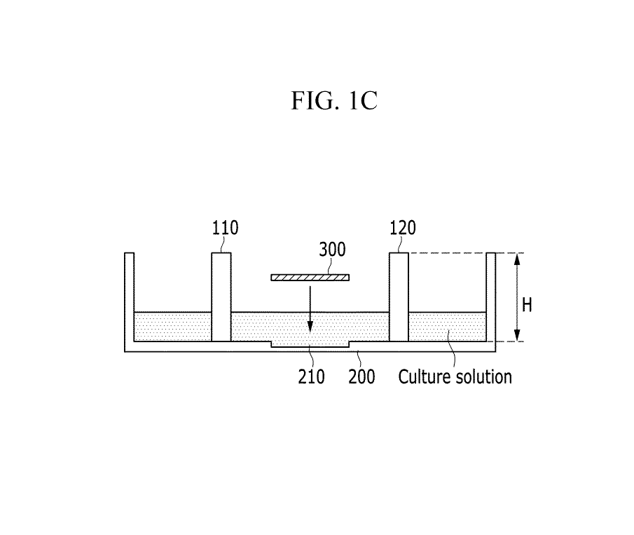

FIGS. 1A to 1C are plan and cross-sectional views illustrating an exemplary embodiment of an apparatus for applying an electric field according to the invention.

As shown in FIGS. 1A to 1C, an exemplary embodiment of an apparatus for applying an electric field according to the invention includes an electrode unit 100, a base 200 and a sample plate 300.

The electrode unit 100 may include a pair of electrodes, e.g., a first electrode 110 and a second electrode 120, facing each other in parallel (e.g., in a horizontal direction), and the pair of electrodes 110 and 120 may be fixed into the base 200. In such an embodiment, the electrode unit 100 forms a substantially uniform electric field between the pair of electrodes 110 and 120 facing each other in parallel. The sample plate 300 is configured to be disposed, e.g., installed, in a predetermined position on the base 200, and the cells may be placed on an upper surface of the sample plate 300. In one exemplary embodiment, for example, the sample plate 300 may include or be made of glass, but is not limited thereto. In an alternative exemplary embodiment, and may be made of any material so long as the sample plate 300 functions as a ‘plate’ on which the cells may be placed.

The following Equation 1 represents uniformity of the electric field formed between the pair of electrodes 110 and 120.

$$Equation 1 \text{:}$$

$$Uniformity = 1 - \frac{∯ \vert E - {E}_{ave} \vert d A}{{E}_{ave} \times {S}_{area}}$$

In Equation 1, E denotes an electric field strength (V/m), Eₐᵥₑ denotes a surface averaged electric field strength, and Sₐᵣₑₐ denotes a surface area of a cover glass of the sample plate 300. In an exemplary embodiment, a numerical analysis method may be used to specify an area of the sample plate 300 and a region in which the uniform electric field is maintained. In such an embodiment, after the distribution of the electric field formed by the pair of electrodes 110 and 120 is calculated through the numerical analysis, a length of the electrode unit 100 may be optimized based on the size of the sample plate 300, or the size of the sample plate 300 may be selected based on the length of the electrode unit 100.

In an exemplary embodiment of the invention, a cuboid electrode having a small width W and a large length L may be used so that the electric field may be substantially uniformly formed in a desired range. In one exemplary embodiment, for example, when the electric field is applied to the cells contained in a solution (e.g., a culture solution), a height H of the electrode unit 100 may be larger than a depth of the solution so that the electric field may be uniformly maintained between the pair of electrodes 110 and 120.

The base 200 may contain a solution such as a culture solution, and the cells placed on the sample plate 300 may be cultured in the solution filled in the base 200. In an exemplary embodiment of the invention, the cells may be placed on the sample plate 300 disposed in a cell region S₁, and the cell region S1 may be defined to be completely included in the electric field applied between the pair of electrodes 110 and 120 of the electrode unit. In such an embodiment, an area S2 between the pair of electrodes 110 and 120 of the electrode unit 100 formed by the length L of the electrode unit 100 and a distance T between the pair of electrodes 110 and 120 is large than the cell region S₁. Since the cell region may be generally determined by the area of the sample plate 300, the length L of the electrode unit 100 and the distance T between the pair of electrodes 110 and 120 may be larger than a length of one side of the sample plate 300. In one exemplary embodiment, for example, the length L of the electrode unit 100 is larger than a length I_{P} of the sample plate 300, or the distance T between the pair of electrodes 110 and 120 may be larger than a width w_{P} of the sample plate 300. In an exemplary embodiment of the invention, a relationship between the length L of the electrode unit 100, the distance T between the pair of electrodes 110 and 120, and the width w_{P} and the length I_{P} of the sample plate 300 is represented as the following Inequality 1.

$$Inequality 1 \text{:}$$

$$1 \leq \frac{T}{{W}_{P}} \leq 2 or 1 \leq \frac{L}{{I}_{P}} \leq 2$$

In an exemplary embodiment of the invention, the sample plate 300 allows the cells to be effectively separated from the base 200. In an exemplary embodiment, where the sample plate 300 has a predetermined size, a groove 210 having substantially the same size (e.g., the same area and shape) as the sample plate 300 may be formed at a portion of the base 200 at which the sample plate 300 is to be disposed, as shown in FIG. 1C. In such an embodiment, the groove 210 formed in the base 200 seats and fixes the sample plate 300, and as a result, the sample plate 300 may be accurately arranged at a portion where the electric field is uniformly formed in the base 200. In such an embodiment, after the cells are cultured on a separate culture dish with a desired density, the cultured cells may be transferred to the apparatus for applying an electric field, and the electric field may be applied to the cultured cells after up to a culture step is completed while the cells are transferred to the apparatus for applying an electric field. An experimenter using an exemplary embodiment of the apparatus for applying an electric field according to the invention may perform a next process by separating only the sample plate 300 from the base 200 after applying the electric field to the cultured cells.

Accordingly, in an exemplary embodiment of the invention, only the cells stimulated through the uniform electric field may be easily separated from the base 200. In the related art, chemical methods such as trypsin processing and physical methods such as centrifugation may be used to separate the culturing cells from the base 200. In an exemplary embodiment of the invention, only the sample plate 300 is separated from the base 200, such that a non-controlled parameter is effectively prevented from being generated.

In an exemplary embodiment of the invention, the electrode unit 100 may be plated with gold or platinum to ensure bio-compatibility, or be formed with at least one of gold, platinum and stainless steel. In an exemplary embodiment, the base 200 may be made of a bio-compatibility ensured material (e.g., polystyrene, glass, crystal, and the like) so that only the electric field may be applied to the cells without another stimulus. In an exemplary embodiment, the base 200 may have a similar shape to a general culture dish, the electric stimulus for a short time of several seconds to the electric stimulus for a long time of several days or more may be applied to the cells.

In such an embodiment, the culture solution is a high conductive solution containing many ions, and when the electric field is applied between the pair of electrodes 110 and 120 by the apparatus for applying the electric field, a temperature of the culture solution rises by joule heating. Generally, when the temperature of the culture solution is about 50° C. or more, a fatal damage to the cells may be caused, and even in the temperature rise of several ° C., a change in a molecular structure of the cells may occur. Heat is typically discharged to a side and an upper side of the base 200 filled with the culture solution by natural convection, a discharging speed is slow, and thus it may be insufficient to prevent the temperature rise. Accordingly, in an exemplary embodiment of the invention, a thickness of the bottom of the base 200 is substantially small, e.g., in a range of about 1 millimeter (mm) to 3 mm, and a cooling device (not shown) such as a heat sink having high thermal capacity may be attached to the lower portion of the base 200. In an exemplary embodiment of the invention, a cooling fin or a cooling fan may be installed in the heat sink to smoothly discharge the heat to the outside. In such an embodiment, the temperature of the culture solution filled in the base 200 may be kept at a predetermined level through forced cooling.

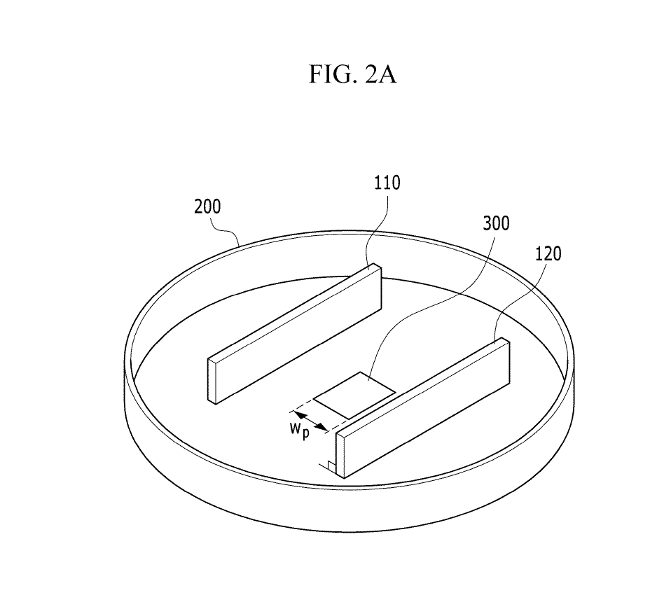

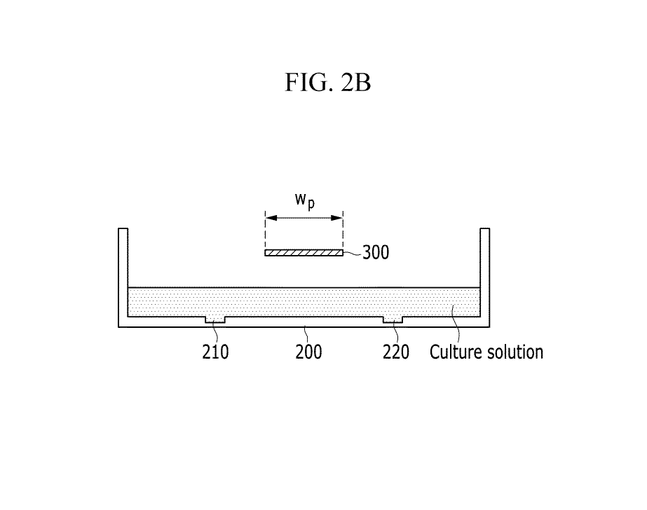

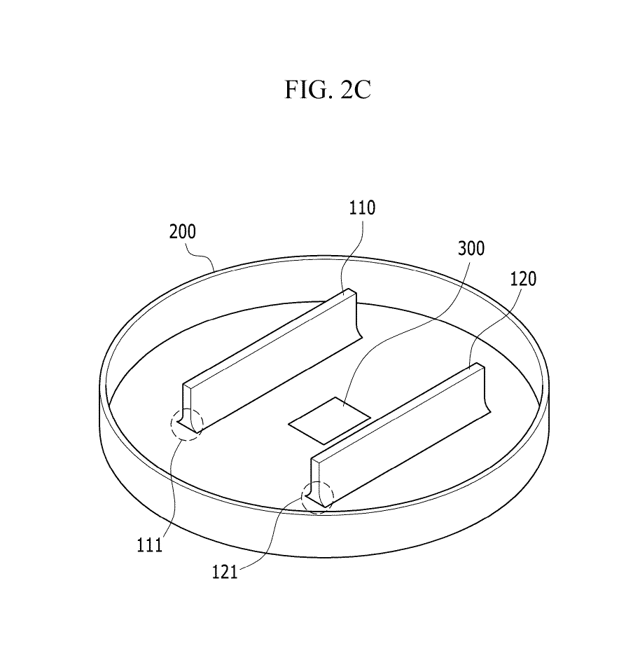

FIGS. 2A to 2C are perspective and cross-sectional views of exemplary embodiments of the apparatus for applying an electric field according to the invention.

Referring to FIGS. 2A and 2B, in an exemplary embodiment, side surfaces (surfaces defined by the directions of lengths and heights) of the pair of electrodes 110 and 120 included in the electrode unit 100 may be vertical to the bottom inside the base 200, that is, an inner bottom surface of the base 200. In one exemplary embodiment, for example, the electrode unit 100 may be disposed to be vertical to the bottom inside the base 200. In such an embodiment, the electrode unit 100 may be fixed to the base 200 by a groove 210 or 220 defined on the bottom inside the base 200 (see FIGS. 2A and 2B).

Referring to FIG. 2C, in an alternative exemplary embodiment, areas of portions 111 and 121 contacting the bottom inside the base 200 may be larger than that of other portions in the electrode unit 100, to allow the electrode unit 100 to stably stand on the bottom inside the base 200 (see FIG. 2C).

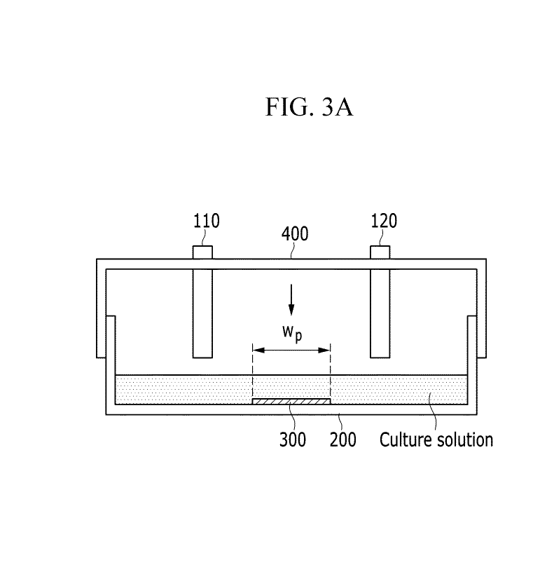

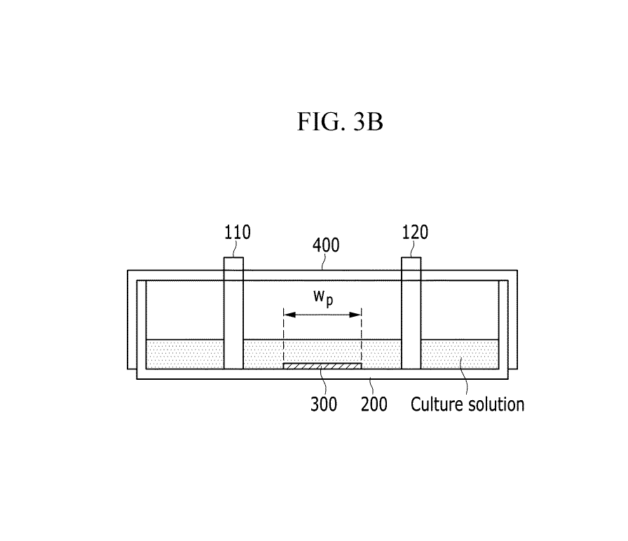

Hereinafter, an exemplary embodiment of the apparatus for applying the electric field including a cover unit will be described with reference to FIGS. 3A and 3B.

FIGS. 3A and 3B are cross-sectional views illustrating another exemplary embodiment of an apparatus for applying an electric field according to the invention.

Referring to FIGS. 3A and 3B, in an exemplary embodiment, the apparatus for applying the electric field may further include a cover unit 400. In such an embodiment, the cover unit 400 may fix the electrode unit 100 thereto so that the electrode unit 100 may be maintained at a predetermined height with respect to the base 200. In one exemplary embodiment, for example, the cover unit 400 may function as a cover for covering an upper surface of the base 200, and the pair of electrodes 110 and 120 may be attached to a surface of the cover unit 400. In such an embodiment, the height H of the electrode unit 100 may be greater than or equal to a height of an internal region of the base 200, e.g., an inner space defined in the cover unit 400 and the base 200 when the cover unit 400 is coupled to the base 200.

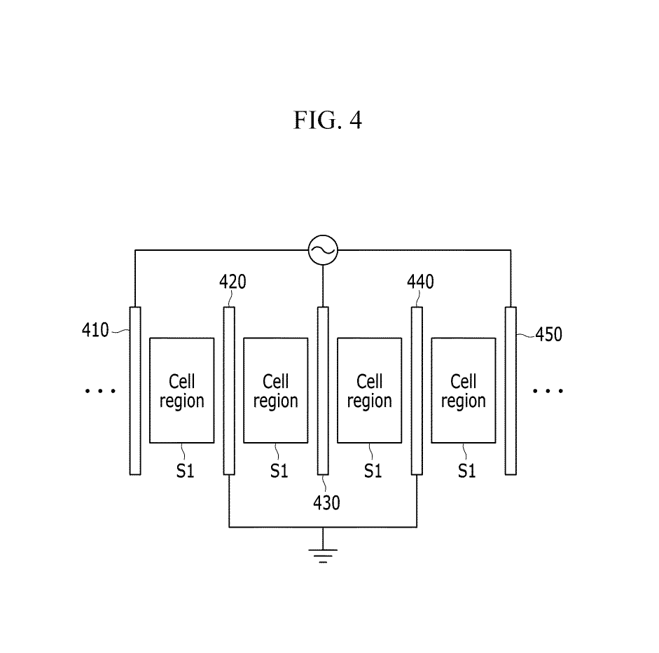

FIG. 4 is a diagram illustrating another exemplary embodiment of the apparatus for applying an electric field according to the invention.

In an exemplary embodiment, the apparatus for applying the electric field may apply a uniform electric field to a plurality of cell regions S₁. FIG. 4 is a schematic plan view of such an embodiment of the apparatus for applying the electric field according to the invention. In one exemplary embodiment, for example, power is applied to odd-numbered electrodes 410, 430 and 450, and even-numbered electrodes 420 and 440 are grounded to generate the uniform electric field between a pair of adjacent electrodes, as shown in FIG. 4.

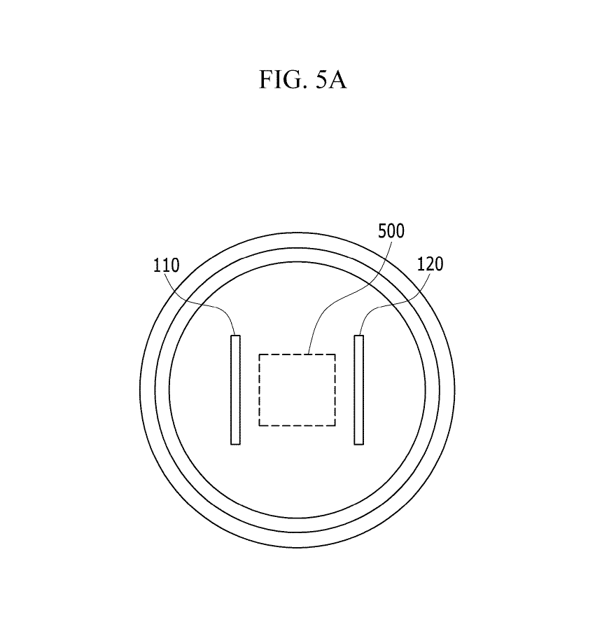

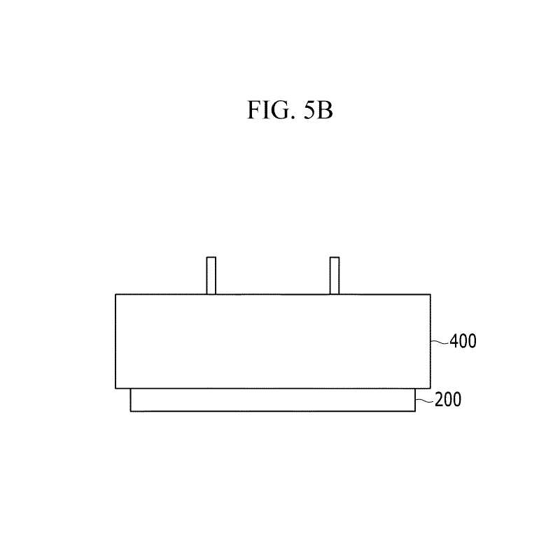

FIGS. 5A and 5B are top and side views of an exemplary embodiment of the apparatus for applying an electric field according to the invention.

Referring to FIGS. 5A and 5B, an exemplary embodiment of the apparatus for applying an electric field according to the invention is an apparatus for applying an electric field in which a cell culture dish having a radius of about 35 millimeters (mm) is set as the base 200. In such an embodiment, a distance between the pair of electrodes 110 and 120 is about 16 mm, and a length of each of the pair of electrodes 110 and 120 is about 15 mm. In such an embodiment, a sample plate 500 is positioned between the pair of electrodes 110 and 120, and a size of the sample plate 500 is about 10 mm by about 10 mm. In such an embodiment, the pair of electrodes 110 and 120 is fixed to the cover unit 400, and a function generator or a pulse generator for forming the electric field may be connected to the electrode unit 100 portion protruding upward of the cover unit 400. In such an embodiment, a commercial groove is formed with a depth of 0.2 mm on a seating surface of the sample plate 500 in the bottom inside the base 200. In such an embodiment, while the electric field is applied by using a groove for an electrode formed on the bottom inside the base 200, the distance between the pair of electrodes 110 and 120 may be substantially stably or uniformly maintained.

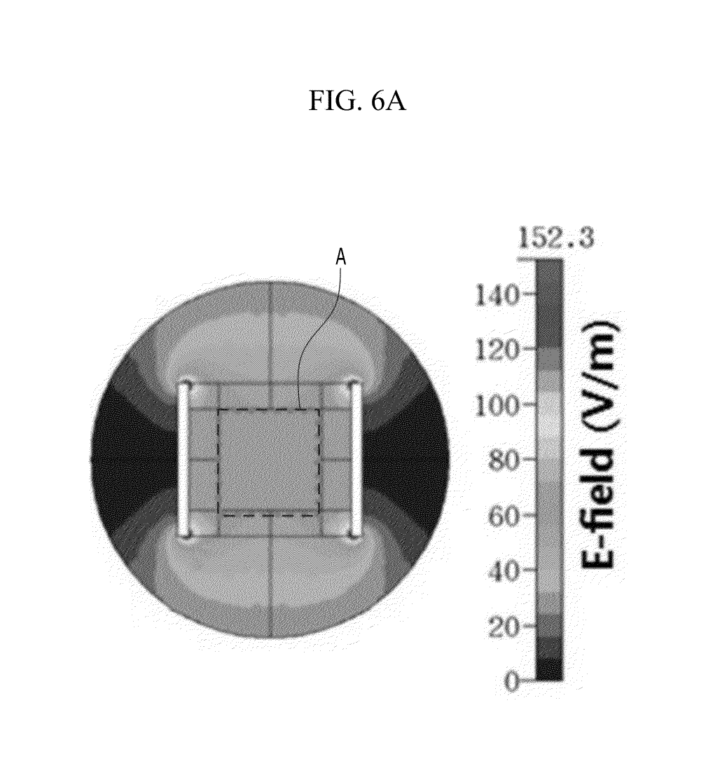

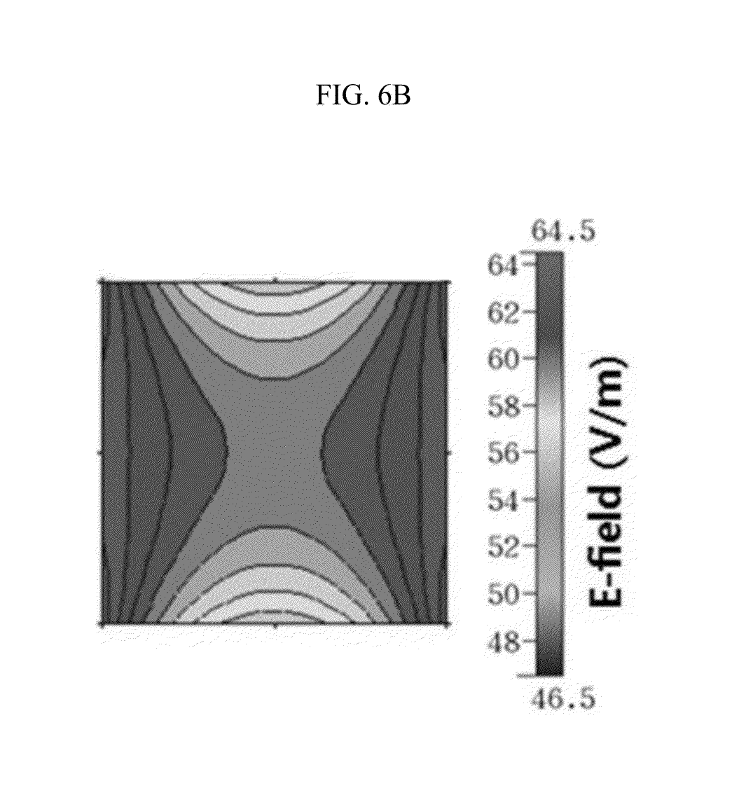

FIG. 6A is a diagram illustrating distribution of the electric field which is applied by an exemplary embodiment of the apparatus for applying an electric field illustrated in FIGS. 5A and 5B, and FIG. 6B is an enlarged view of the portion A in FIG. 6A.

FIGS. 6A and 6B illustrate the distribution of the electric field in a case where a potential difference of about 1 volt (V) is applied between the pair of electrodes, and uniformity of the electric field distribution is 97.8%.

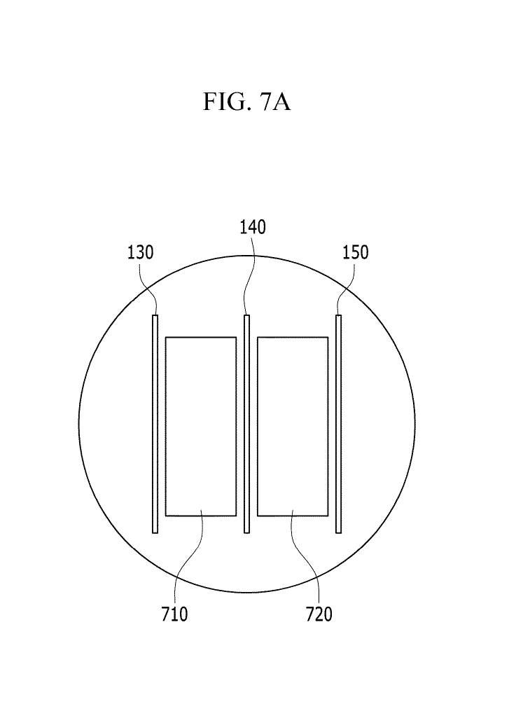

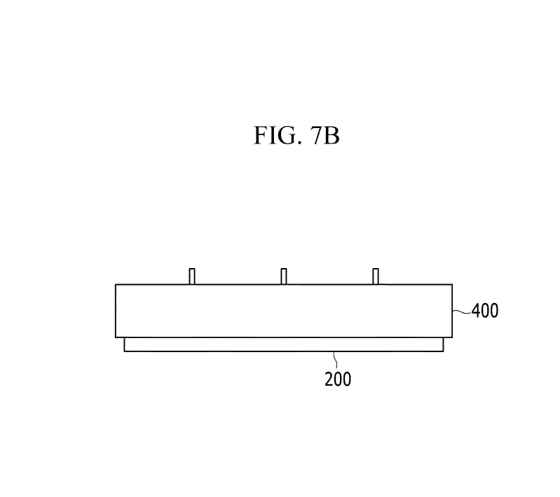

FIGS. 7A and 7B illustrate plan and front views of another exemplary embodiment of the apparatus for applying an electric field according to the invention.

Referring to FIGS. 7A and 7B, an exemplary embodiment of the apparatus for applying an electric field according to the invention is an apparatus for applying an electric field in which a cell culture dish having a radius of about 100 mm defines the base 200. In such an embodiment, a distance between adjacent electrodes among a plurality of electrodes, e.g., a first electrode 130, a second electrode 140 and a third electrode 150, is about 28 mm, and a length of each of the electrodes 130, 140 and 150 is about 70 mm. In such an embodiment, each of two sample plates 710 and 720 may be positioned between adjacent electrodes of the electrodes 130, 140 and 150, and a size of each of the sample plates 710 and 720 is about 22 mm×60 mm. In such an embodiment, the three electrodes are fixed to the cover unit 400, and a function generator or a pulse generator for applying the electric field may be connected to the electrodes 130, 140, and 150 portions protruding upward of the cover unit 400.

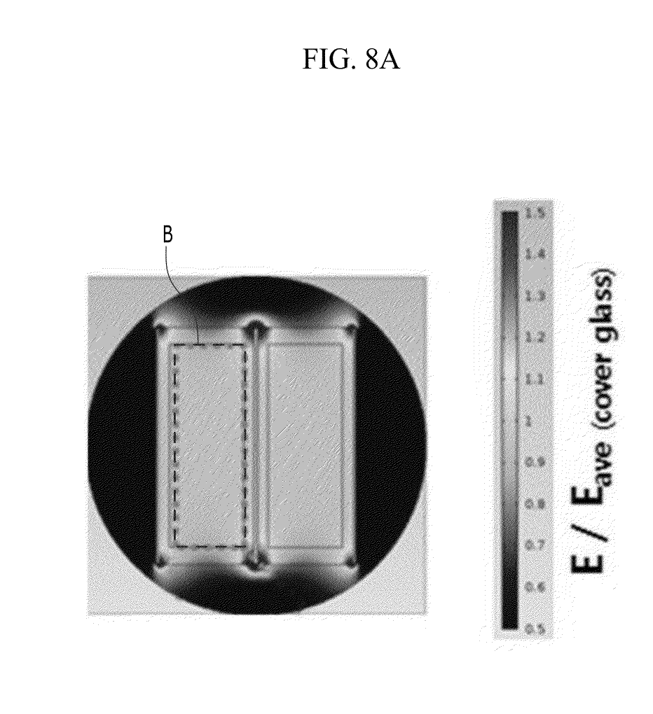

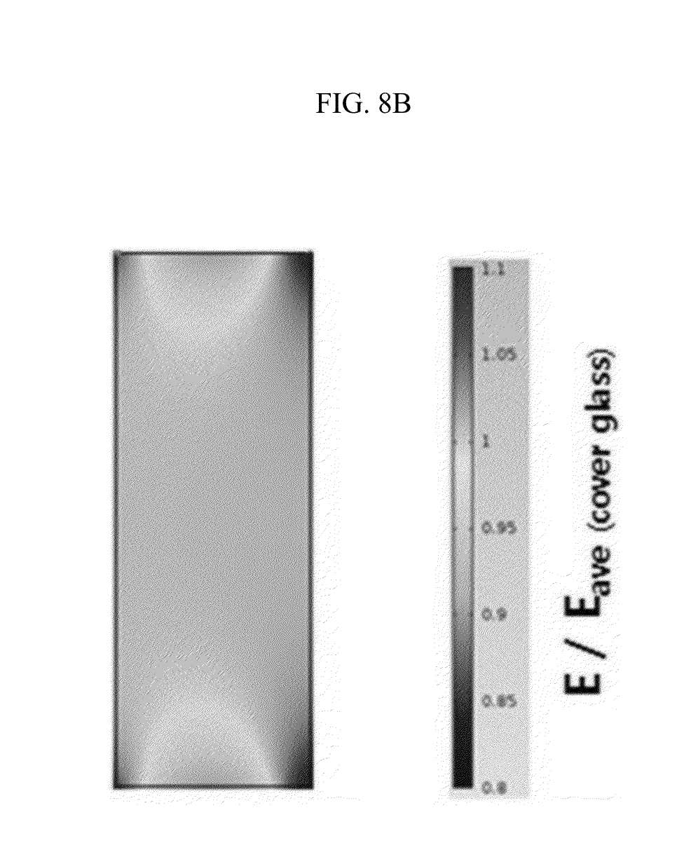

FIG. 8A is a diagram illustrating distribution of the electric field which is applied by an exemplary embodiment of the apparatus for applying an electric field illustrated in FIGS. 7A and 7B, and FIG. 8B is an enlarged view of the portion B in FIG. 8A.

FIGS. 8A and 8B illustrate the distribution of the electric field in a case where a potential difference of about 1 V is applied between the electrodes, and uniformity of the electric field distribution is about 98.8%.

As described herein, an exemplary embodiment of the apparatus for applying an electric field according to the invention may be used to apply uniform electric field to the cells. In such an embodiment, after the electric field is applied, the cells may be stably separated from the base 200, and the uniform electric field may be applied for a predetermined or desired time without a time limit. In such an embodiment, the apparatus for applying an electric field may simultaneously apply the electric field to a plurality of cell regions using a plurality of electrodes that may be effectively provided therein, such that an operation with the apparatus may be effectively expanded and the temperature rise of the culture solution by joule heating may be effectively suppressed.

While the invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. An apparatus for applying an electric field, comprising:
a base, on which an internal region is defined;
a plurality of electrodes disposed to face each other in parallel in the internal region of the base; and
a sample plate disposed between the plurality of electrodes.

2. The apparatus of claim 1, wherein uniformity of the electric field formed between the plurality of electrodes is about 90% or more.

3. The apparatus of claim 1, wherein the plurality of electrodes has a cuboid shape.

4. The apparatus of claim 1, wherein
the plurality of electrodes comprises three or more electrodes, and
the sample plate comprises two or more sample plates.

5. The apparatus of claim 1, wherein
a relationship between a distance between the plurality of electrodes and a width of the sample plate satisfies the following inequality: 1≦T/W≦2,
wherein
T denotes a distance between two adjacent electrodes of the plurality of electrodes, and
W denotes the width of the sample plate disposed between the two adjacent electrodes.

6. The apparatus of claim 1, wherein a relationship between a length of each of the plurality of electrodes and a length of the sample plate satisfies the following inequality: 1≦L/I≦2,
wherein
L denotes the length of each of the plurality of electrodes, and
I denotes the length of the sample plate.

7. The apparatus of claim 4, wherein
a groove is defined on the base, and
the sample plate is disposed in the groove.

8. The apparatus of claim 1, wherein
a plurality of grooves is defined on the base, and
the plurality of electrodes is disposed in the plurality of grooves, respectively.

9. The apparatus of claim 1, wherein the base comprises polystyrene, polycarbonate, glass, or crystal with ensured bio-compatibility.

10. The apparatus of claim 1, wherein the plurality of electrodes is plated with gold or platinum.

11. The apparatus of claim 1, wherein the plurality of electrodes comprises at least one of gold, platinum and stainless steel.

12. The apparatus of claim 1, further comprising:
a cover unit which fixes the plurality of electrodes thereto and is coupled to the base such that the plurality of electrodes is maintained at a predetermined height with respect to the base when the cover unit is coupled to the base,
wherein the height of the plurality of electrodes is greater than or equal to the height of the internal region of the base.

13. The apparatus of claim 1, further comprising:
a cooling device which discharges heat in the internal region of the base to outside.

14. The apparatus of claim 13, wherein the cooling device comprises a heat sink disposed below the base.
